(19) United States
(12) Patent Application Publication
(10) Pub. No.: US 2026/0260702 A1
(43) Pub. Date: Sep. 3, 2026

(54) Methods And Systems For Enhancing Gene Translation

(71) Applicant: Monsanto Technology LLC, St. Louis, MO (US)

(72) Inventors: Aabid SHARIFF, Durham, NC (US); Krishnakumar SRIDHARAN, Ballwin, MO (US); Jose Juan Tapia VALENZUELA, St. Louis, MO (US)

(21) Appl. No.: 17/012,849

(22) Filed: Sep. 4, 2020

Related U.S. Application Data
(60) Provisional application No. 62/896,470, filed on Sep. 5, 2019.

(51) Int. Cl. G16B 25/10

(52) U.S. Cl. CPC G16B 25/10

(57) ABSTRACT

Example methods for use in providing gene translation and protein expression are disclosed. One example method includes, for an input coding sequence, identifying a feature of the coding sequence as a candidate for change, based on an effect of the feature on a score for the coding sequence; selecting, by a conversion engine computing device, a codon of the coding sequence associated with the identified feature; inserting a codon equivalent for the selected codon, wherein the codon equivalent is based on a temperature associated with the coding sequence; calculating a score for the coding sequence with the codon equivalent and a score for the input coding sequence; and advancing the coding sequence with the codon equivalent to a next iteration when the score for the coding sequence with the codon equivalent indicates an enhancement relative to the score of the input coding sequence.

### CROSS-REFERENCE TO RELATED APPLICATION

This application claims the benefit of, and priority to, U.S. Provisional Application No. 62/896,470, filed on Sep. 5, 2019. The entire disclosure of the above application is incorporated herein by reference.

### FIELD

The present disclosure generally relates to methods and systems for use in plant breeding, and in particular to methods and systems for enhancing gene translation and protein expression, through a high-throughput, automated process of altering codons in a coding sequence in a gene of interest.

### BACKGROUND

This section provides background information related to the present disclosure which is not necessarily prior art.

A principle of molecular biology includes sequential transfer of information from DNA to RNA to proteins. Codons are triplet DNA sequences (or complementary RNA sequences) which code for particular amino acids. A sequence of codons encodes a particular amino acid sequence of a protein. The genetic code is degenerate, in that most amino acids are encoded by multiple codons. For example, four different codons code for glycine. Thus, it is possible to alter the underlying nucleic acid sequence of DNA (and, consequently, the corresponding RNA) without changing the amino acid sequence of the protein. Despite the direct correlation between a DNA sequence and its corresponding protein, the level of transcription from DNA to RNA does not necessarily lead to an equivalent amount of the translated protein. It is possible that while transcription of a given gene is high, the concomitant protein expression may be low. This can occur for various reasons, including messenger RNA (mRNA) misfolding, undesirable post-transcriptional modification of the mRNA (e.g., improper splicing, 5′ capping does not occur, or an insufficient poly-A tail, etc.), or post-transcriptional silencing (e.g., via miRNA, etc.). Types of mRNA misfolding and/or undesirable sequences include suboptimal CG/AT content, motif removal, miRNA sites, inverted repeats, RNA secondary structure (e.g., hairpin loops, etc.), and Kozak sequences. Each of these misfolding and/or undesirable sequences lead to premature or unregulated breakdown and removal of the mRNA, destabilizing the transcript, and/or silencing the mRNA, all of which result in lowered protein expression.

Previous publications have identified the potential causes of low gene expression in transgenic plants. Conventional modifications to a genome are made in a cell or plant in a deliberate, stepwise manner. The gene is modified using site-directed mutagenesis. Cells and/or plants containing modified genes of interest are then assayed individually. To the extent that a particular parameter is identified (e.g., GC content, etc.), the identification occurs on a global level, and does not account for the specific position within the gene. U.S. Pat. No. 5,500,365, for example, identifies a particular motif known to cause low steady state levels of mRNA, resulting in reduced gene expression for the gene of interest. In the '365 patent, the inventors use in planta techniques to genetically modify plants to reduce the prevalence of the unwanted motif, thereby leading to enhanced gene translation due to improved stability of the mRNA. With that said, such traditional methods of genetic engineering is time, labor, and resource intensive, in part because each mutation may need to be physically, individually assayed for the desired effect.

### DRAWINGS

The drawings described herein are for illustrative purposes only of selected embodiments, are not all possible implementations, and are not intended to limit the scope of the present disclosure.

FIG. 1 illustrates an example system of the present disclosure suitable for use in providing enhanced gene translation and protein expression;

FIG. 2 is a block diagram of a computing device that may be used in the example system of FIG. 1;

FIGS. 3A-C illustrate an example method, which may be implemented in connection with the system of FIG. 1, for providing enhanced gene translation and protein expression; and

FIGS. 4A-F illustrate another example method, which may be implemented in connection with the system of FIG. 1, for providing enhanced gene translation and protein expression.

### DETAILED DESCRIPTION

Example embodiments will now be described more fully with reference to the accompanying drawings. The description and specific examples included herein are intended for purposes of illustration only and are not intended to limit the scope of the present disclosure.

Protein expression depends, in part, on the efficiency of gene transcription (DNA→RNA) and gene translation (RNA→protein). Genes with high transcription may have low ultimate protein expression due to factors like post transcriptional processing of the messenger RNA (mRNA). For example, the mRNA transcript may develop a secondary structure (e.g. a hairpin loop, etc.) that slows or blocks translation. Alternatively, the mRNA transcript may contain a sequence that binds microRNA (miRNA or μRNA), which initiates degradation of the mRNA, thereby silencing the gene. In both instances, protein expression is reduced. Uniquely, the systems and methods described herein allow for identification of undesired mRNA sequences and alteration of the plant genome to reduce or eliminate presence of the undesired mRNA sequences. In other embodiments, the systems and methods described herein allow for the alteration of transgene sequences to reduce or eliminate presence of the undesired mRNA sequences, thus optimizing transgene expression in planta. The modification to the plant genome or transgene sequence takes advantage of the degenerate genetic code, where changing one or two base pairs does not affect the final amino acid sequence of the encoded protein. In particular, the systems and methods identify particular proteins of interest (e.g., a protein that leads to a desirable phenotype but has relatively low expression, etc.) and the mRNA a plant cell used to encode that protein. The mRNA sequence is examined for, among other things, GC content, the presence of deleterious motifs, miRNA target sites, inverted repeats (IRs), RNA secondary structures, and Kozak sequences. The mRNA transcript is queried on a codon-by-codon basis, accounting for position of the codon within the transcript, to determine whether a change to one or more particular codons improves gene translation. The systems and methods then may change the nucleotide composition of one or more codons within the gene of interest based on a codon frequency table (e.g., changing a codon for valine from GUG to GUA, etc.), for example, to generate an mRNA sequence that has an improved sequence, such as a preferable GC content and/or a reduction in deleterious motifs, miRNA target sites, inverted repeats, RNA secondary structures and/or Kozak sequences, etc. The systems and methods output multiple variants for in vitro and in vivo testing to validate the expected improvement and/or enhancements in gene translation and/or protein expression.

As used herein, “GC content” refers to the relative number of guanine (G) and cytosine (C) nucleotides in a given DNA or RNA sequence as compared to the total number of bases in the entire sequence, which also includes adenine (A) and thymine (T; for DNA) or uracil (U; for RNA). GC content is strongly indicative of high performance in plants (e.g., where high GC content prevents transgenes from silencing, increase transgene expression, etc.). Optimal GC content leads to more stable mRNA transcripts and, consequently, more stable protein expression levels. The present systems and methods facilitate selection of synonymous alternative codons to bring the GC content within a desirable range without changing the resulting protein sequence. The desirable range of GC content can be supplied by the user, the art, or determined by the process.

As used herein, a “deleterious motif” refers to genetic motifs which negatively affect mRNA transcript stability and expression. The deleterious motifs include structural motifs, polyadenylation sites, undesired cleavage sequences, far upstream elements, 5′- and 3′-destabilizing sequences, transcription factor binding sites, undesired splice sites, and restriction enzyme sites. These motifs can be supplied by the user or the art. The deleterious motifs are identified and removed from the coding sequence by exchanging an offending codon with a synonymous codon such that the resulting protein sequence is unaffected.

As used herein, “miRNA target sites” refers to mRNA motifs corresponding to known plant miRNA. miRNA binds to a miRNA target site on the mRNA, resulting in a double helix RNA molecule, which targets the mRNA for degradation by endogenous cellular machinery. The miRNA target sites, supplied by the user or the art, are identified and removed from the coding sequence by exchanging an offending codon with a synonymous codon such that the resulting protein sequence is unaffected.

As used herein, “inverted repeats” (IR) refers to an mRNA transcript with a palindromic sequence, which may or may not be interrupted by a spacer. An example of an inverted repeat is 5′-ACUGGnnnnCCAGU-3′ (where n is any nucleotide). An mRNA comprising an inverted repeat can bind to itself due to the base pairing to form destabilizing hairpin loops. Such sequences are identified and removed from the coding sequence by exchanging an offending codon with a synonymous codon such that the resulting protein sequence is unaffected.

As used herein, “RNA secondary structure” refers to a predicted or actual three dimensional RNA structure resulting from the oligonucleotide sequence. Undesirable secondary structures can reduce stability of the mRNA transcript, thereby reducing the overall expression of the protein. Sequences that lead to the undesired RNA secondary structures are identified and removed from the coding sequence by exchanging an offending codon with a synonymous codon such that the resulting protein sequence is unaffected.

As used herein, “Kozak sequences” refer to sequences that initiate translation from RNA to protein. Kozak sequences may induce translation at the wrong site of the mRNA transcript, leading to truncated proteins having reduced or no function. Sequences corresponding to the putative Kozak sequence, and obvious variants thereof, are identified and removed from the coding sequence by exchanging an offending codon with a synonymous codon such that the resulting protein sequence is unaffected. Further, as used herein, “temperature” or T should be understood to include a statistical term in the context of the description below (and thus not a physical property), etc.

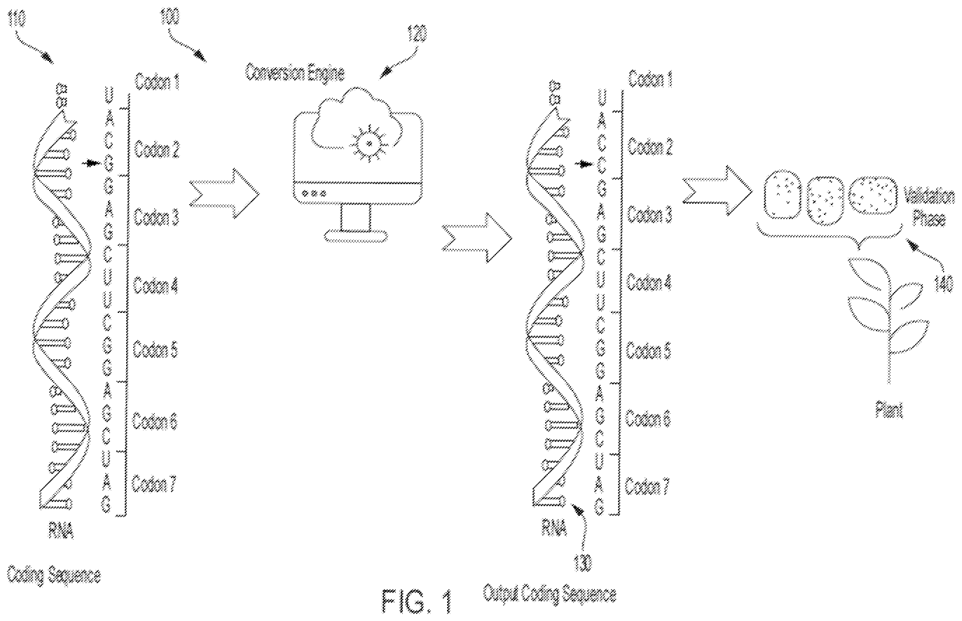

With that said, FIG. 1 illustrates an example system 100 for enhancing gene translation and protein expression, in which one or more aspects of the present disclosure may be implemented. Although, in the described embodiment, parts of the system 100 are presented in one arrangement, other embodiments may include the same or different parts arranged otherwise depending, for example, on the sequential iteration(s) to introduce genetic changes, etc.

As shown in FIG. 1, the system 100 generally includes a coding sequence 110, which corresponds to a specific gene of interest and a particular protein. The coding sequence 110 includes a succession of letters indicating an order of nucleotides forming the alleles within the RNA of the gene of interest. Each group of three letters, or bases, is referred to herein as a codon. The codons include, for example, as shown, ACG, GAG, etc. Further, in this example embodiment, the coding sequence is associated with corn or maize plants. That said, it should be appreciated that the system 100 may be employed for other plants, including, without limitation, soybeans, etc. What's more, the gene of interest may include, without limitation, proteins that confer insect resistance, proteins that confer herbicide resistance, proteins that confer disease resistance, etc. It should further be appreciated that other suitable genes of interest and/or proteins may be included as the gene of interest in system 100, including, for example, reporter genes such as GUS, etc. Moreover, the present disclosure may also be used in gene-editing efforts, etc. In some embodiments the gene of interest is a transgene. In other embodiments the gene of interest is an endogenous gene.

The system 100 also includes a conversion engine 120, which may be integrated and/or implemented in a computing device (such as computing device 200 described in detail hereinafter), or in a network-based service hosted by one or more computer devices. In either case, the conversion engine 120 is configured, by executable instructions, to affect the processes and/or operations herein to operate as described.

The system 100 further includes a validation phase 140, which is configured to provide for testing of an output coding sequence 130, in vitro and/or in vivo, whereupon the output coding sequence 130 is transformed, through various processes, into one or more plant seeds (or other organisms (e.g., bacteria, etc.)) for further validation, scientific use, and/or commercial use, etc.

In the context of the system 100, the coding sequence 110 is enhanced by operation of the conversion engine 120. In particular, the conversion engine 120 is configured to iteratively modify the coding sequence 110, where specific codons are replaced with alternate codons based on a codon equivalence (or frequency) table (thereby preserving the amino acid(s)). In connection therewith, the conversion engine 120 is configured to score the coding sequence for each iteration, in which the sequence is modified, and to select the best performing coding sequence(s) as the output coding sequence 130. The iterative process is generally based on a variance in the temperature, which is a proxy for stability in this context (and, more particularly, stability of an optimization algorithm search, as described in more detail hereinafter). In general, an annealing schedule (also known as a cooling schedule, and also potentially referred to as a temperature schedule) is provided, which progresses from a high temperature to a lower temperature, thereby imposing low stability to high stability (i.e., a lower temperature may limit the number of equivalent codons) on the iterative process.

In particular, the conversion engine 120 is configured to identify inverted repeats, miRNA target sites, and deleterious motifs (and other undesirable features) within the coding sequence 110 and to score the coding sequence 110. Such scoring may be done, for example, based on Equation 1, presented below (where E′ generally represents an energy term). Or, alternatively, it may be done based on Equation 6 or Equation 7 discussed hereinafter.

$${E}^{′} = 100 * μ RNA + 2.5 * IR + f ( Motifs ) \qquad ( 1 )$$

In Equation 1, it should be appreciated that a weighting expressed, per term, impacts the overall score for the coding sequence 110, whereby the weighting is indicative of the importance of a given term (the same also applies for Equations 6 and 7). It should also be appreciated that different weighting or even equations may be employed to score the coding sequence (or iterations thereof), other than what is provided in Equation 1 (see, e.g., Equations 6 and 7 below, etc.). For example, one or more of the same or different “features” of the coding sequence may be accounted for in scoring the coding sequence at one or more different weights, to affect the score differently. With that said, in general, Equation 1 (and Equations 6 and 7) uses the energy term as a way of penalizing a sequence for local features/subsequences that may or may not be present (e.g., motifs, IRs, miRNA target sites, etc.). As such, GC content is not accounted for in the example Equation 1 (or in Equations 6 or 7). Instead, GC content is relied on as a global feature, such that it is adjusted separate from Equation 1. More particularly, for every iteration, Equation 1 relies on the precondition that the sequence meets the GC content criteria, and when necessary, modifies as many random codons as necessary to adjust the overall sequence GC content (e.g., adjusting a glycine from GGT to GGC may increase the GC content of the overall sequence, etc.). It should be appreciated that, in other embodiments, the energy term may instead account for all constraints a sequence must meet (whether they are local or global). In so doing, GC restrictions may be addressed in an explicit, incremental way rather than implicitly and all at once (whereby efficiency of the algorithm used in determining the energy term may be improved).

Thereafter, the conversion engine 120 is configured to iteratively modify the coding sequence according to an annealing schedule. In particular, the conversion engine 120 is configured to modify the coding sequence, in multiple iterations, based on equivalent codons as defined by a first temperature in the annealing schedule. For example, for each iteration of a temperature, the conversion engine 120 is configured to adjust the GC content of the coding sequence 110 to be within a defined threshold (e.g., between 57% and 60%, etc.) and then to remove miRNA target sites in the coding sequence 110, to remove inverted repeats in the coding sequence 110, and to remove deleterious motifs from the coding sequence 110 (and, potentially, to remove other undesirable features of the coding sequence). Each is accomplished, again, by randomly swapping one or more codons with alternate, but equivalent codons (based on temperature) (e.g., according to a weighted random distribution, etc.). Then, for each iteration, the conversion engine 120 is configured to identify inverted repeats, miRNA target sites, and deleterious motifs within the modified coding sequence 110 and to score the coding sequence 110, based on Equation 1.

The conversion engine 120 is configured to then compare the score of the original coding sequence 110 and the score(s) of one or more of the iterations of the coding sequence 110 (having the alternate codons). When the score is improved for the new coding sequence, the conversion engine 120 is configured to feed the new coding sequence into the next iteration of Equation 1 (or Equations 6 or 7) as the starting coding sequence.

In this example embodiment, when the score of the new coding sequence is not improved (based on the score), the conversion engine 120 is configured to use Equation 2, below, to compare the scores of the iteration(s) to the score of the original coding sequence or prior new coding sequence. The conversion engine 120 is configured to then generate a random threshold (e.g., between 0 and 1, etc.) and to compare the probability (p) of the new coding sequence (as defined by Equation 2) to the random threshold. When the probability satisfies the random threshold (e.g., is above the random threshold, etc.), the conversion engine 120 is configured to feed the new coding sequence into the next iteration as the starting coding sequence. When the probability does not satisfy the random threshold, the conversion engine 120 is configured to discard the new coding sequence and feed the prior coding sequence into the next iteration as the starting coding sequence.

$$p = exp ( - \frac{{E}^{′} - E}{T} ) \qquad ( 2 )$$

Notwithstanding the direct comparison of the resulting scores from Equations 1, 6, or 7 and the specific probability function of Equation 2 described above (for use in determining which coding sequence to implement in the next iteration), it should be appreciated that the scores may be compared otherwise in other example embodiments to make the same determination.

After each iteration at the specific temperature is completed (or stops), the conversion engine 120 is configured to advance to a next temperature in the annealing schedule and to repeat the iterative modification and scoring of the coding sequence. It should be appreciated that, in doing so in some embodiments, the conversion engine 120 may be configured to employ parallel processing to modify and score the coding sequence as part of the multiple different iterations at one time, whereby the iterations vary, for example, as defined by the annealing schedule. In this manner, the conversion engine 120 may provide a parallel tempering algorithm (e.g., in connection with replica exchange Markov chain Monte Carlo (MCMC) sampling, etc.), whereby multi-objective enhancements of the gene(s) of interest may be achieved (as generally described in connection with method 400).

Next in the system 100, after the iterations are complete for each temperature in the annealing schedule, the conversion engine 120 is configured to identify one or more coding sequences, based on the scores, as described above, as the output sequence 130. The output coding sequence(s) 130 is then provided to the validation phase 140, wherein the output coding sequence(s) 130 is realized and tested. The enhanced gene of interest is then converted into RNA, by a person or automated process, as generally known to those skilled in the art.

And, in some embodiments, in the validation phase 140, the DNA encoding the RNA is transformed, by person or process, into a plant or plant cell by conventional means known in the art. For example, a transgene encoding the RNA of interest can be inserted into Agrobacterium, which then introduces the genetic material to plant tissue. The transformed plant tissue is then cultured in appropriate medium to promote root formation. Upon shoot formation, the transgenic plant is transferred to the appropriate soil for validation of the desired phenotype or gene of interest. Alternatively, the genetic material can be introduced into a plant cell via a gene gun, electroporation, zinc finger nucleases (ZFNs), transcription activator-like effector nucleases (TALENs), or the CRISPR/Cas9 system, or the CRISPR/Cpf1 system, etc. In other embodiments, in the validation phase 140, genomic DNA is edited to encode the output coding sequence.

Notwithstanding the above, it should be appreciated that the conversion engine 120 may be configured otherwise to employ a different manner of modifying and scoring the coding sequence, as indicated below. For example, in some embodiments the conversion engine 120 may employ a simulated annealing process as generally described in connection with method 300.

In one example embodiment, in connection with parallel tempering, the conversion engine 120 is configured to initialize the input coding sequence 110 into a number of different chains, where each chain is associated with a different temperature in a given annealing schedule (or as selected randomly). The conversion engine 120 is configured to then initialize each chain, in parallel, for a desired number of iterations.

Specifically, for each chain, and iteration thereof, the conversion engine 120 is configured to identify a feature (or issue) of the coding sequence to be altered (e.g., a deleterious motif, inverted repeat, miRNA target site, etc.), where the specific feature may include a feature having a highest negative impact in scoring of the coding sequence. Once identified, the conversion engine 120 is configured to change one or more codons affecting the identified feature, as limited or indicated by the given temperature selected for the chain (as associated with the available codon equivalents). The conversion engine 120 is configured to then identify each feature or issue within the coding sequence 110 and to score the coding sequence 110. For this example embodiment, the conversion engine 120 may be configured to employ Equation 6 or Equation 7, as presented below (e.g., instead of Equation 1 above (although Equation 1 may be used in other embodiments), etc.). The conversion engine 120 is configured to then compare a score for the original coding sequence 110 (or the prior evaluated coding sequence) and the score of the changed coding sequence, and to advance the higher scoring coding sequence (or a randomly selected lesser scoring coding sequence, as described above, based on application of Equation 2, etc.) into a next iteration. When a number of iterations is completed, the coding sequence from the last iteration is identified and advanced.

Next, the conversion engine 120 is configured to swap the coding sequences for the chain with coding sequences from other ones of the chains, and then to repeat the iterations. The swap exposes certain coding sequences to alteration under a different temperature constraint, which is potentially randomly selected (or alternatively associated with the given annealing schedule). The conversion engine 120 is configured to then expose the coding sequence of each chain, at the new temperature, to further iterations, in parallel (in generally the same manner as described above). After a predefined number of iteration and swaps between chains, the conversion engine 120 is configured to identify one or more coding sequences, based on the scores, as described above, as the output sequence 130. The output coding sequence(s) 130 is then provided to the validation phase 140, wherein the output coding sequence(s) 130 is realized and tested. The enhanced gene of interest may then be converted into RNA, by a person or automated process, as generally known to those skilled in the art. And, in the validation phase 140, a transgene encoding the RNA is transformed, by person or process, into a plant or plant cell, as described above. In other embodiments, in the validation phase 140, genomic DNA is edited to encode the enhanced gene of interest.

Additionally, or alternatively, it should be appreciated that more or less changes to the coding sequence may be accomplished through the conversion engine 120, as described herein. Specifically, for example, the conversion engine 120 may be configured to add one or more Kozak sequences to the coding sequences above as desired. What's more, the conversion engine 120 may be configured to add or remove RNA structures from the coding sequences above and/or to implant one or more sequence motifs or small RNA binding sites into the coding sequences herein.

In connection therewith, several embodiments of the present disclosure relate to a recombinant nucleic acid comprising one or more output coding sequences. As used herein, a “recombinant nucleic acid” refers to a nucleic acid molecule (DNA or RNA) having a coding and/or non-coding sequence distinguishable from endogenous nucleic acids found in natural systems. In some aspects, a recombinant nucleic acid provided herein is used in any composition, system or method provided herein. In some aspects, a recombinant nucleic acid provided herein is a transgene. In some aspects, a recombinant nucleic acid may encode any protein and can be used in any composition, system or method provided herein. In some embodiments, a recombinant nucleic acid comprises one or more output coding sequences operably linked to a heterologous promoter. In one aspect, a recombinant nucleic acid provided herein comprises one or more, two or more, three or more, four or more, five or more, six or more, seven or more, eight or more, nine or more, or ten or more heterologous promoters operably linked to one or more, two or more, three or more, four or more, five or more, six or more, seven or more, eight or more, nine or more, or ten or more output coding sequences.

In some aspects, a recombinant nucleic acid may be contained in a vector. As used herein, the term “vector” refers to a nucleic acid molecule capable of transporting another nucleic acid to which it has been linked. Vectors include, but are not limited to, nucleic acid molecules that are single-stranded, double-stranded, or partially double-stranded; nucleic acid molecules that comprise one or more free ends, no free ends (e.g., circular, etc.); nucleic acid molecules that comprise DNA, RNA, or both; and other varieties of polynucleotides known in the art. One type of vector is an Agrobacterium T-DNA. Another type of vector is a viral vector, wherein virally-derived DNA or RNA sequences are present in the vector for packaging into a virus (e.g., retroviruses, replication defective retroviruses, Tobacco mosaic virus (TMV), Potato virus X (PVX) and Cowpea mosaic virus (CPMV), tobamovirus, Gemini viruses, adenoviruses, replication defective adenoviruses, and adeno-associated viruses, etc.). Viral vectors also include polynucleotides carried by a virus for transfection into a host cell. In some embodiments, a viral vector may be delivered to a plant using Agrobacterium. Certain vectors are capable of autonomous replication in a host cell into which they are introduced. Other vectors are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein. In some embodiments, an expression vector can comprise one or more output coding sequences in a form suitable for expression of the output coding sequences in a plant cell, which means that the expression vector comprises one or more regulatory elements that are operatively-linked to the output coding sequences to be expressed. Regulatory elements may include enhancers, termination sequences, introns, etc.

In certain embodiments, one or more output coding sequences may include or be operably linked to a nucleic acid sequence encoding one or more target peptides (for example: nuclear localization signals (NLS), nuclear export signals (NES), chloroplast transit peptides (CTP), plastid transit peptides, etc.), functional domains, flexible linkers, etc. Target peptides have the ability to transport a protein (in this instance, the protein encoded by the output coding sequence) to a specific cellular location (these locations could be the nucleus, mitochondria, endoplasmic reticulum (ER), chloroplast, apoplast, peroxisome, plasma membrane, etc.) The one or more of the target peptides or the functional domain may be conditionally activated or inactivated. In particular embodiments it can be of interest to target the protein encoded by the output coding sequence to the chloroplast. For example, this targeting may be achieved by including or operably linking the output coding sequence encoding the protein of interest to a nucleic acid encoding a chloroplast transit peptide (CTP) or plastid transit peptide. Other options for targeting to the chloroplast which have been described are the maize cab-m7 signal sequence (U.S. Pat. No. 7,022,896, WO 97/41228, incorporated by reference herein), a pea glutathione reductase signal sequence (WO 97/41228, incorporated by reference herein), and the CTP described in US 2009/029861, incorporated by reference herein.

In an aspect, a vector provided herein comprises any recombinant nucleic acid comprising an output coding sequence as provided herein. In another aspect, a plant cell provided herein comprises a recombinant nucleic acid provided herein. In another aspect, a plant cell provided herein comprises a vector provided herein. The plant cell may be of a monocot or dicot. In some embodiments, the plant cell may be from or of a crop or grain plant such as cassava, corn, sorghum, alfalfa, cotton, soybean, canola, wheat, oat or rice. The plant cell may also be of an algae, tree or production plant, fruit or vegetable (e.g., trees such as citrus trees, (e.g., orange, grapefruit or lemon trees; peach or nectarine trees; apple or pear trees; etc.); nut trees such as almond or walnut or pistachio trees; nightshade plants; plants of the genus Brassica; plants of the genus Lactuca; plants of the genus Spinacia; plants of the genus Capsicum; cotton, tobacco, asparagus, avocado, papaya, cassava, carrot, cabbage, broccoli, cauliflower, tomato, eggplant, pepper, lettuce, spinach, strawberry, potato, squash, melon, blueberry, raspberry, blackberry, grape, coffee, cocoa, etc.).

That said, and as generally described above, numerous methods for transforming chromosomes or plastids in a plant cell with a recombinant DNA molecule are known in the art, which can be used according to the systems and methods of the present application to produce a plant cell and plant comprising one or more output coding sequences (e.g., at the validation phase 140, etc.).

In planta, particle bombardment or biolistic delivery can be used for delivering recombinant nucleic acids. Particle bombardment is suitable to transform plants with DNA, RNA, protein, or any combinations thereof. Methods of transforming plants using biolistic delivery of DNA is described in PCT/US2019/033984 and incorporated by reference herein, in its entirety.

In planta, Agrobacterium mediated transformation is a suitable method of choice for delivering recombinant nucleic acids on one or more T-DNAs. Agrobacterium mediated transformation is widely applied to monocot and dicot species. The expression cassettes comprising one or more output coding sequences may be provided, in one embodiment, as double tumor-inducing (Ti) plasmid border constructs that have the right border (RB or AGRtu.RB) and left border (LB or AGRtu.LB) regions of the Ti plasmid isolated from Agrobacterium tumefaciens comprising a T-DNA that, along with transfer molecules provided by the A. tumefaciens cells, permit the integration of the T-DNA into the genome of a plant cell (see, e.g., U.S. Pat. No. 6,603,061, which is incorporated herein by reference in its entirety). The constructs may also contain the plasmid backbone DNA segments that provide replication function and antibiotic selection in bacterial cells, e.g., an Escherichia coli origin of replication such as ori322, a broad host range origin of replication such as oriV or oriRi, and a coding region for a selectable marker such as Spec/Strp that encodes for Tn7 aminoglycoside adenyltransferase (aadA) conferring resistance to spectinomycin or streptomycin, or a gentamicin (Gm, Gent) selectable marker gene. In some embodiments, one or more expression cassettes comprising one or more output coding sequences are provided in a T-DNA binary vector that has a low copy origin of replication, such as the OriRi vector backbone. For plant transformation, the host bacterial strain is often A. tumefaciens ABI, C58, or LBA4404, however other strains known to those skilled in the art of plant transformation can function in the present disclosure. In some embodiments, an Agrobacterium tumefaciens strain that lacks certain DNA recombination functions, such as RecA, is utilized to deliver expression vectors encoding CAST system components to plant cells.

In some embodiments, the expression cassettes comprising one or more output coding sequences as described herein are provided on a single T-DNA. In some embodiments, the expression cassettes encoding one or more output coding sequences as described herein are provided on multiple separate T-DNAs and delivered to plant cells in a single transformation process, or in separate sequential transformation processes.

Several embodiments relate to a plant comprising in its genome an output coding sequence as described herein. In certain embodiments, genome editing methods are utilized for the modification or replacement of an existing genomic coding sequence, such as a coding sequence for a protein conferring herbicide tolerance, within a plant genome with a sequence encoding an output coding sequence as provided by the methods described herein. In some embodiments, the native genomic coding sequence is modified to comprise one or more targeted nucleotide changes, additions, deletions, or other modifications. Several embodiments relate to the use of a known genome editing methods, and a site-specific genome modification enzyme, such as zinc-finger nucleases, engineered or native meganucleases, TALE-endonucleases, or an RNA-guided endonucleases (for example, a Clustered Regularly Interspersed Short Palindromic Repeat (CRISPR)/Cas9 system, a CRISPR/Cpf1 system, a CRISPR/CasX system, a CRISPR/CasY system, a CRISPR/Cascade system) to modify or replace an existing coding sequence in the genome of a plant. Several embodiments relate to providing a site-specific genome modification enzyme capable of recognizing a specific gene of interest within a genome of a plant to allow for alteration of the native sequence by non-templated editing or by templated editing. Several embodiments relate to providing a base modification agent (such as a deaminase) linked to a site-specific genome modification enzyme capable of recognizing a specific nucleotide sequence of interest within a genome of a plant to allow for alteration of the native sequence by the base modification agent to provide the output coding sequence.

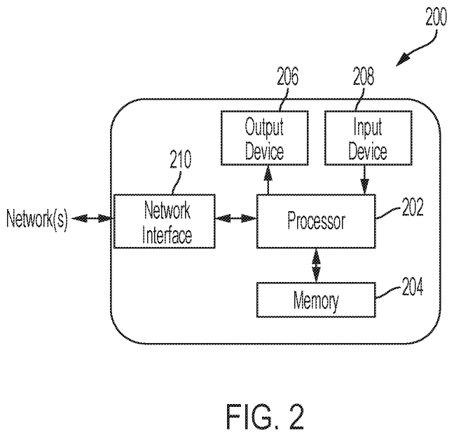

FIG. 2 illustrates an example computing device 200 that can be used in the system 100. The computing device 200 may include, for example, one or more servers, workstations, personal computers, laptops, tablets, smartphones, PDAs, terminals, etc. In addition, the computing device 200 may include a single computing device, or it may include multiple computing devices located in close proximity or distributed over a geographic region, so long as the computing devices are specifically configured to function as described herein. In the system 100 of FIG. 1, the conversion engine 120 is illustrated and described as including, or being implemented in, a computing device 200. That said, the system 100, or parts thereof, should not be understood to be limited to the computing device 200, as other computing device may be employed in other system embodiments. In addition, different components and/or arrangements of components may be used in other computing devices.

Referring to FIG. 2, the example computing device 200 includes a processor 202 and a memory 204 coupled to (and in communication with) the processor 202. The processor 202 may include one or more processing units (e.g., in a multi-core configuration, etc.). For example, the processor 202 may include, without limitation, a central processing unit (CPU), a microcontroller, a reduced instruction set computer (RISC) processor, an application specific integrated circuit (ASIC), a programmable logic device (PLD), a gate array, and/or any other circuit or processor capable of the functions described herein.

The memory 204, as described herein, is one or more devices that permit data, instructions, etc. to be stored therein and retrieved therefrom. The memory 204 may include one or more computer-readable storage media, such as, without limitation, dynamic random access memory (DRAM), static random access memory (SRAM), read only memory (ROM), erasable programmable read only memory (EPROM), solid state devices, flash drives, CD-ROMs, thumb drives, floppy disks, tapes, hard disks, and/or any other type of volatile or nonvolatile physical or tangible computer-readable storage media. The memory 204 may be configured to store, without limitation, temperature data structures, deleterious motifs, miRNA target sites, initialization parameters, scoring algorithms, coding sequences, codon equivalence data structures, codon frequency data structures, and/or other types of data (and/or data structures) as needed and/or suitable for use as described herein. Furthermore, in various embodiments, computer-executable instructions may be stored in the memory 204 for execution by the processor 202 to cause the processor 202 to perform one or more of the functions described herein, such that the memory 204 is a physical, tangible, and non-transitory computer readable storage media. Such instructions often improve the efficiencies and/or performance of the processor 202 that is performing one or more of the various operations herein (e.g., one or more of the operations of method 300, method 400, etc.), whereby the computing device 200 may be transformed into a special-purpose computing device. It should be appreciated that the memory 204 may include a variety of different memories, each implemented in one or more of the operations or processes described herein.

In the example embodiment, the computing device 200 includes an output device 206 (or presentation unit) that is coupled to (and that is in communication with) the processor 202 (however, it should be appreciated that the computing device 200 could include output devices other than the output device 206, etc.). The output device 206 outputs information (e.g., output coding sequences, scores, etc.), either visually or audibly, to a user of the computing device 200, for example, a user associated with the conversion engine 120, a user associated with the validation phase 140, etc. Various interfaces (e.g., as defined by network-based applications, etc.) may be displayed at computing device 200, and in particular at output device 206, to display such information. The output device 206 may include, without limitation, a liquid crystal display (LCD), a light-emitting diode (LED) display, an organic LED (OLED) display, an “electronic ink” display, speakers, etc. In some embodiments, output device 206 includes multiple devices.

The computing device 200 also includes an input device 208 that receives inputs from the user (i.e., user inputs) such as, for example, selection of a gene of interest, etc., or inputs from other computing devices. The input device 208 is coupled to (and is in communication with) the processor 202 and may include, for example, a keyboard, a pointing device, a touch sensitive panel (e.g., a touch pad or a touch screen, etc.), another computing device, and/or an audio input device. Further, in various example embodiments, a touch screen, such as that included in a tablet, a smartphone, or similar device, behaves as both output device 206 and input device 208.

In addition, the illustrated computing device 200 also includes a network interface 210 coupled to (and in communication with) the processor 202 and the memory 204. The network interface 210 may include, without limitation, a wired network adapter, a wireless network adapter, a mobile network adapter, or other device capable of communicating to/with one or more different networks. Further, in some example embodiments, the computing device 200 includes the processor 202 and one or more network interfaces incorporated into or with the processor 202.

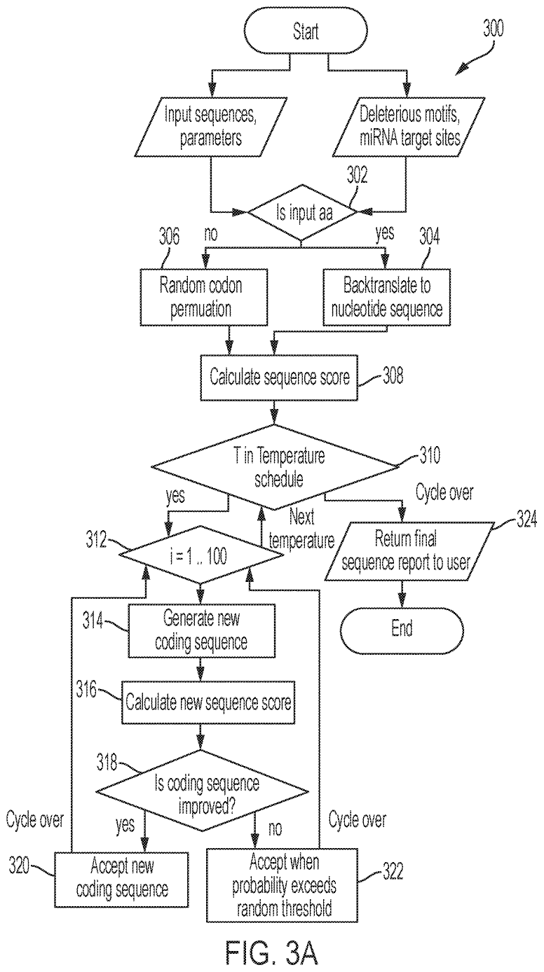

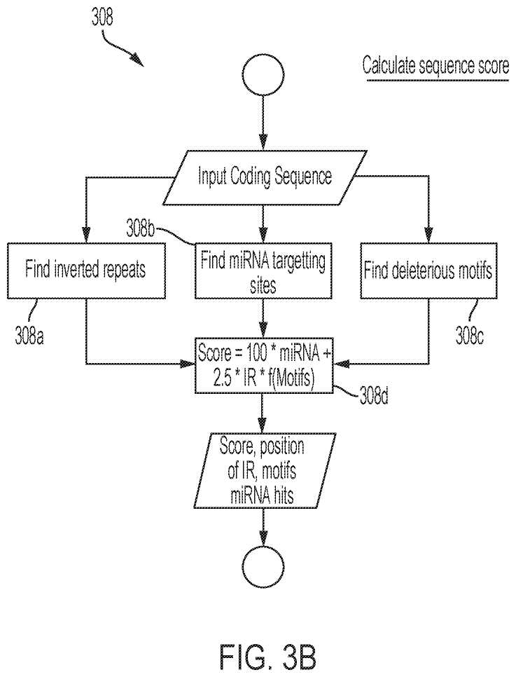

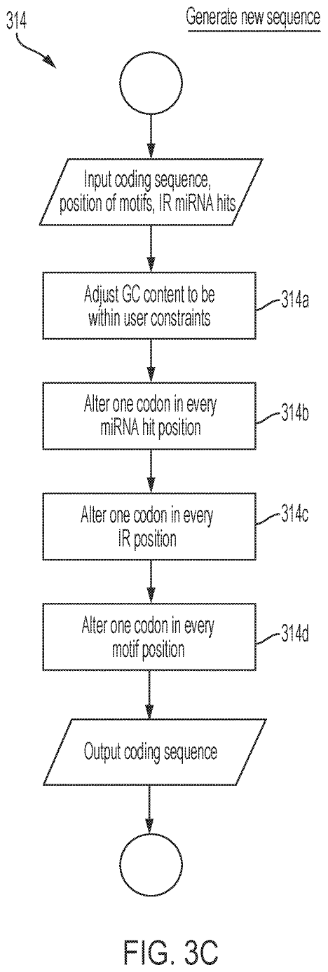

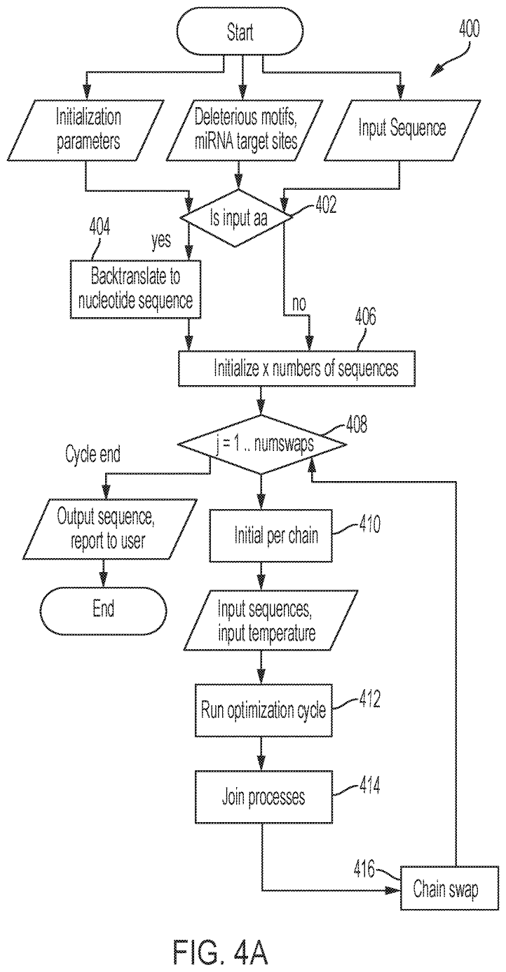

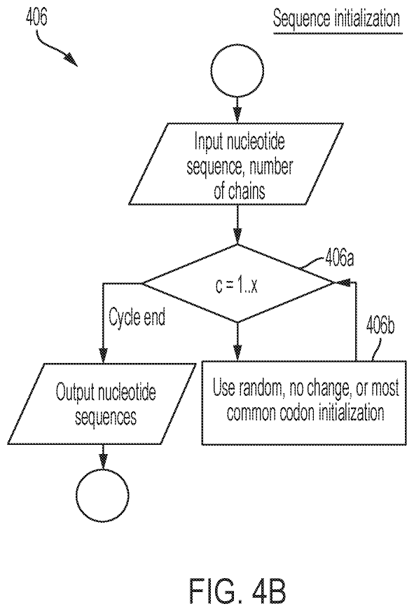

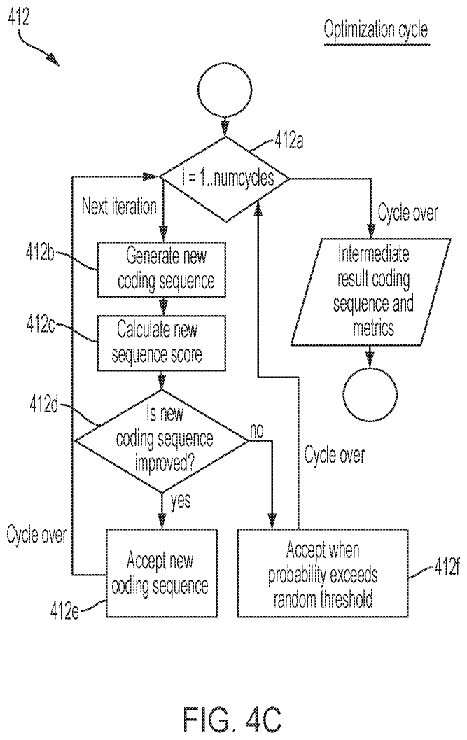

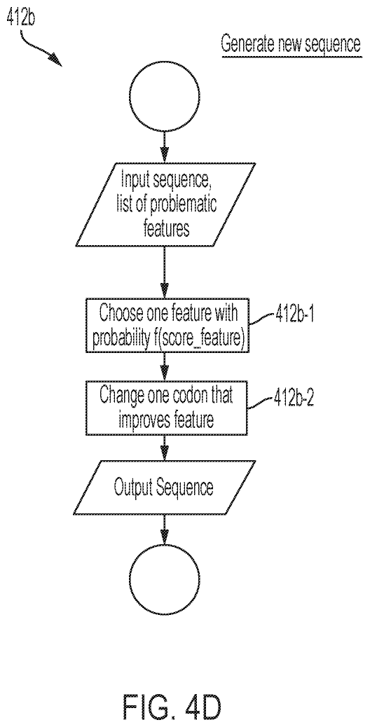

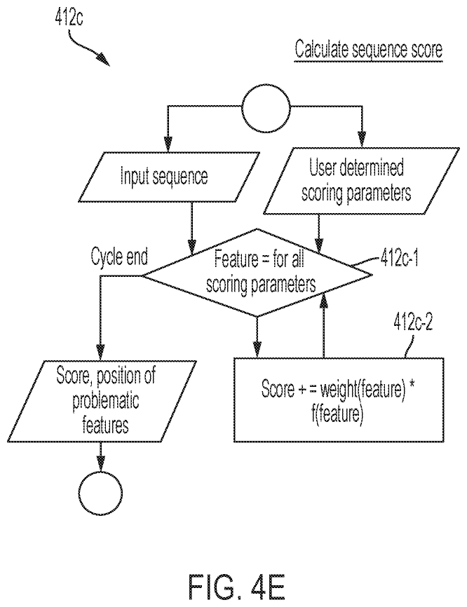

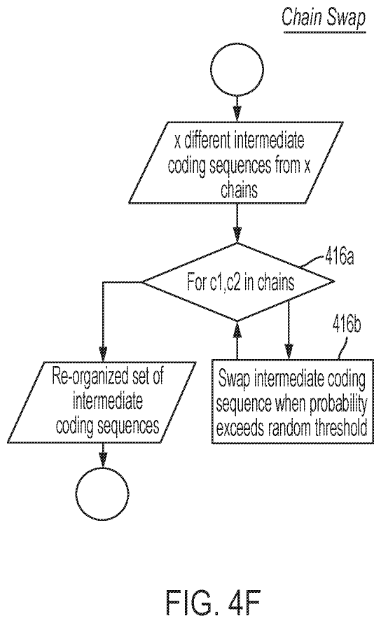

FIGS. 3 and 4 relate to different example methods for use in enhancing translation and protein expression for a gene of interest. Each of the example methods includes modification of a coding sequence through an iterative processes and a selection of one or more output coding sequences based on scoring associated with the iterative coding sequences. That said, each is different in the manner and/or interaction among the different iterations, as described in more detail below.

Specifically, the method 300 is provided as a simulated annealing process, while the method 400 is provided as a parallel tempering process. In general, simulated annealing is a stochastic optimization technique that includes a stage of parameter exploration in which a large diversity of parameter configurations can be evaluated, followed by a stage of parameter exploitation in which a small set of the parameter configurations are identified that performed well (in order to further improve on the identified configurations). Through use of the exploration and exploitation stages, simulated annealing, as implemented in method 300, may enhance an algorithm's ability to efficiently converge on a desired solution.

Parallel tempering, as described with reference to method 400, provides another approach of utilizing such exploration and exploitation stages, by simultaneously executing the stages in parallel threads and allowing the stages to exchange information about the parameter landscape they have discovered, for example, at set intervals. For instance, parallel tempering may provide N copies (or chains) of the coding sequence, randomly initialized at different temperatures. Each one of these copies of the coding sequence is referred to as a chain or replica. And, the method 400 makes coding sequences as modified at higher temperatures available to lower temperature modifications, and vice versa, whereby the method 400 simultaneously explores low performing regions using the high temperature chains and exploits high performing regions using the low temperature chains (e.g., as a manner of refining the regions, etc.). Consequently, method 400 improves the performance of the algorithm by increasing the quantity and quality of information each chain sees and/or has available thereto. What's more, the method 400 employs codon position-specific frequency tables rather than global codon frequency tables (which may also be employed in method 300, while permitting new features (e.g., regulatory sequences, etc.) to be imparted in the coding sequences. This is described in more detail hereinafter.

That said, FIGS. 3A-C illustrate example method 300 for use in enhancing translation and protein expression for a gene of interest by way of the simulated annealing process. The method 300 is described below in connection with the example system 100 of FIG. 1 and the example computing device 200 of FIG. 2. However, it should be appreciated that the method 300 is not limited to the system 100 or the computing device 200, but may be implemented in a variety of different systems and/or computing devices. Likewise, the systems and computing devices described herein should not be understood to be limited to the example method 300, or other methods described herein.

In connection with FIG. 3A, the method 300 is initiated by accessing a data structure including coding sequence parameters, and also including deleterious motifs and miRNA target sites (e.g., in memory 204 of a computing device 200 in communication with the conversion engine 120, etc.), whereby the same is available to the conversion engine 120 for use as described below. Example coding sequence parameters may include, without limitation, a specific GC content, instructions to remove inverted repeats and miRNA target sites, identification of deleterious motifs to be removed, and annealing schedules for the modifications by the conversion engine 120. The accessed data structure also includes the codon listings for the specific deleterious motifs, miRNA target sites, etc. In connection therewith, specifically, the data structure may include, with regard to the deleterious motifs: polyadenylation sites, transcription factor binding sites, splicing sites, restriction sites, destabilizing motifs, etc.

Additionally, a codon equivalence and frequency table may be accessed by the conversion engine 120 (e.g., for use as input to any of Equations 1, 6, or 7 to be used herein, etc.) (e.g., during backtranslation, in selecting alternate codons, etc.). As part thereof, the conversion engine 120 or other computing device may be configured to generate the table. In particular, the conversion engine 120 may be configured to build codon frequency tables for individual codon positions or ranges of codon positions in the input coding sequence(s), by, for example, taking into account a range of positions relative to the 5′ end of the coding sequence, taking into account a range of positions relative to the 3′ end of the coding sequence, and creating the codon frequency table for those positions that are not close to the 5′ or 3′ ends of the coding sequence. The codon frequency table may be compiled otherwise in other embodiments. In connection therewith, for different segments in the sequence, the codon frequency table may not only include a listing of equivalent codons, but also a listing or designation of the most common codons encoding an amino acid. For example, in looking to modify amino acid #1 located somewhere in the beginning of a sequence that has alternate codons 1, 2 and 3, a fully random approach may not distinguish between the codons and may result in selection of any of the codons with the same probability. However, it should be appreciated that the codon frequency table may permit the conversion engine 120 to select, for instance, codon 2 if it is determined that codon 2 is the most frequent codon for amino acid #1 at the beginning of the sequence. Codon 1 or 3 may still be selected, but with a smaller probability.

Also in the method 300, and for example illustration herein, the gene of interest may include the enzyme Cpf1, which is an enzyme suited to enable gene editing efforts, as certain levels of expression of the enzyme are needed to enable targeted editing in plants, etc. In the particular description below, the enzyme Cpf1 is converted, by the conversion engine 120, in connection with a maize use-case for enhanced expression. In connection therewith, and consistent with the above, the example implementation of the method 300 generally includes accessing the following coding sequence parameters/data (e.g., from the data structure, etc.): (a) GC content above 57% and below 60%; (b) a codon frequency table (e.g., for corn, etc.); (c) data relating to removing inverted repeats; (d) data relating to removing miRNA target sites; (e) data relating to removing deleterious motifs; (f) an annealing schedule for simulated annealing: T (temperature)=(10, 5, 3, 1, 0.1, 0.01); and (g) parameters by which each temperature cycle is run for about one hundred iterations. It should be appreciated that these features, parameters, etc. may be different in other embodiments.

As shown in FIG. 3A, after accessing the above data, the conversion engine 120 determines whether the input to the method 300 is an amino acid (aa), or not, at 302. In general, the conversion engine 120 determines if the input is a coding sequence 110 or another representation of the gene of interest. When the input is not a coding sequence (i.e., is an amino acid), the conversion engine 120 backtranslates the input (i.e., an amino acid sequence) into a nucleotide equivalent, at 304, which then is a coding sequence. The backtranslation may include, for a maize codon distribution, for example, selecting which codon is most likely to appear at a given position in the sequence based on the codon frequency table for maize. That said, it should be appreciated that the maize codon frequency table may determine the most common codon, a randomized selection of the codon, or it may rely on a weighted random distribution that is biased towards the most common codon. Conversely, if the input is a coding sequence 110, the conversion engine 120 effects, at 306, a random codon permutation. In other words, for each position, the conversion engine 120 selects an alternate codon using a weighted random distribution.

In this example, because the input already includes the coding sequence 110 for the enzyme Cpf1, backtranslating is not necessary.

Thereafter in the method 300, the conversion engine 120 calculates a sequence score for the coding sequence 110, at 308. FIG. 3B illustrates a sub-process for such calculation, whereby the conversion engine 120 calculates the score for the coding sequence 110 (received as the input). Specifically in the sub-process, the conversion engine 120 receives the coding sequence (as the input) and then identifies the inverted repeats, at 308a, identifies the miRNA target sites, at 308b, and identifies deleterious motifs, at 308c. In so doing, the conversion engine 120 also compiles a data structure, such as, for example, the data structure in Table 1, which includes the number of inverted repeats, deleterious motifs and miRNA target sites identified. Next, the conversion engine 120 employs, at 308d, in this example, Equation 7 below to calculate a score for the coding sequence 110 (where the calculation is generally similar to that described below with regard to method 400). Specifically, the coding sequence 110 for the enzyme Cpf1 is characterized by the data included in Table 1, whereby based on such data Equation 7 may provide an example score of 9.8. That said, it should be appreciated that Equation 1 may be used to perform such scoring in other embodiments.

TABLE 1

| Metric | Input Coding Sequence 110 |
|---|---|
| Deleterious motifs | 785 deleterious motif hits |
| Inverted repeats | 203 inverted repeat hits |
| miRNA target sites | 0 miRNA target site hits |

The sub-process of FIG. 3B for calculating the sequence score for the coding sequence 110 ends by providing/generating the score for the coding sequence 110, and also the position of each of the inverted repeats, motifs and miRNA target sites included (or remaining) in the coding sequence 110. In connection with this operation, tracking the position of the inverted repeats, the miRNA target sites, and the deleterious motifs (broadly, the issues) may permit directly identifying such position when generating new coding sequences, as described below.

Referring again to FIG. 3A, next in the method 300, the conversion engine 120 initiates an iterative process for the coding sequence 110.

In particular, the conversion engine 120 determines that an iteration for the coding sequence is to be performed and retrieves, at 310, one of the temperatures (T) from the temperature data structure (e.g., in memory 204, etc.) (e.g., a N-th temperature, etc.), where the temperatures may include 10, 5, 3, 1, 0.1, 0.01 (as dimensionless units) (e.g., whereby, as used, the temperature may be a dimensionless measure of the exploration versus exploitation ratio of the search model; etc.). With a temperature value of 10, for example, which impacts the available equivalents for codons, the conversion engine 120 initiates, at 312, multiple iterations (e.g., iterations (i)=1 to 100, etc.) of modifying the coding sequence 110 for the given temperature selection. Specifically, to start, the conversion engine 120 initially generates a new sequence, at 314, which is based on the coding sequence 110 and the selected N-th temperature.

FIG. 3C illustrates a sub-process of generating the new sequence (at 314) for the coding sequence 110, for each of the iterations of analysis. In the sub-process of FIG. 3C, starting with the coding sequence 110, the conversion engine 120 adjusts, at 314a, the GC content of the coding sequence 110, by substituting codons within the coding sequence 110 with equivalent codons from a codon equivalence table (or standard codon table, etc.) (e.g., derived based on codon degeneracy (where multiple codons can encode for the same amino acid), etc.). It should be appreciated that this is a condition of the input coding sequence at least for the first iteration (i=1), such that in at least the first iteration, the adjustments at 314a will be either none or limited. A sufficient number of substitutions, as directed by the conversion engine 120, will be performed in order to put the GC content between the 57% and 60% threshold, as identified in the coding sequence parameters for the example enzyme Cpf1.

Next, at 314b, the conversion engine 120 alters one codon (with an equivalent codon) in one or more of the identified miRNA target site positions in the coding sequence 110. The conversion engine 120 also alters one codon (with an equivalent codon) in one or more of the inverted repeat positions in the coding sequence 110, at 314c, and further alters one codon (with an equivalent codon) in one or more of the identified deleterious motif positions in the coding sequence 110, at 314d.

In connection with these alterations or swaps at 314b-c, the codon equivalence table is employed to permit the swap of one codon in the miRNA target site positions and in the deleterious motif positions (broadly, again, features or issues) with another equivalent codon for the amino acid. Where multiple codons are involved in the issue and/or multiple equivalent codons are available, the conversion engine 120 may employ a random selection between the available codons to be swapped into the coding sequence. That is, for example, if a deleterious motif is six nucleotides long, that is, it has 2 codons, any one of its two codons could be altered or modified, resulting in two possible codons to be swapped, which, for example, each have three different equivalent codons from which to select. As such, in this example, six different options may exist to disrupt the deleterious motif, one of which is then selected at random. Stated plainly, in the example embodiment, the specific codon and the equivalent codon, for each of the miRNA target site, the inverted repeat, and/or the deleterious motif, may be selected at random and swapped into the new coding sequence.

It should be appreciated that additionally, or alternately, the conversion engine 120 may rely of the frequency and/or common occurrence of the codons in the amino acid (in general or for a specific position) (e.g., as indicated in the codon equivalence and frequency table, etc.) to swap codon(s) in the new coding sequence in steps 314b-d.

What's more, in connection with altering the coding sequence at 314b-d, the conversion engine 120 may rely on issue tracking to directly identify the positions of issues in the coding sequence. For instance, the codons may not be randomly chosen. Instead, the particular positions of the miRNA target sites, and other issues (IRs and deleterious motifs), may be quickly identified whereby the particular regions identified may be disrupted through swapping of codons with equivalent codons. In addition, by tracking positon positions of the inverted repeats, the miRNA target sites, and the deleterious motifs, the conversion engine 120 may be able to identify which issues were not removed, and if desired, to track the issues through multiple iterations. What's more, as a feedback mechanism to a user, persistent issues may be relied on in connection with application of Equation 7 (during scoring) by adjusting weights in the energy function and determining which issues are more important to remove, etc.

It should be appreciated that while the steps 314b-d are ordered in a particular manner in FIG. 3C, the steps 314b-d may be ordered differently in other embodiments (or even performed in parallel) and/or omitted in still other embodiments.

With continued reference to FIG. 3C, when the steps 314b-d are completed, the conversion engine 120 returns an output coding sequence (e.g., a modified version of the coding sequence 110, etc.) to the method 300 of FIG. 3A, into step 316.

In turn, the conversion engine 120 calculates, at 316, a sequence score for the output (or new) coding sequence from the sub-process of FIG. 3C. To do so, the conversion engine 120 employs the sub-process in FIG. 3B, consistent with the description above for step 308. When the sequence score is returned, the conversion engine 120 then determines, at 318, based on the score for the resulting coding sequence for the iteration and the score for the input coding sequence (from 308), whether the resulting coding sequence is enhanced over the input coding sequence 110. When the score of the new coding sequence is higher than the score of the input coding sequence, for example, the new coding sequence is accepted, at 320, saved in memory (e.g., the memory 204, etc.) and returned to step 312 as the input coding sequence for the next iteration (e.g., i=2, etc.).

Conversely, when the score for the new coding sequence is less than the score of the input coding sequence, the conversion engine 120 applies Equation 2 above to determine a probability between the scores, based on the temperature, and then compares the probability to a randomly generated threshold (e.g., a threshold between 0 and 1, etc.). That said, other equations (other than Equation 2) may be employed in other embodiments to perform such an analysis. When the probability satisfies the random threshold, the new coding sequence is accepted, at 322, saved in memory (e.g., the memory 204, etc.), and returned as the input coding sequence for the next iteration (e.g., i=2, etc.). Conversely, when the probability does not satisfy the random threshold, the new coding sequence is rejected, at 322, and the prior input coding sequence is reused as the input coding sequence for the next iteration (e.g., i=2, etc.). In this manner, a lower score coding sequence has the opportunity to advance as a starting coding sequence for the next iteration, thereby including flexibility in the method 300.

When each of the iterations is complete (e.g., when i=100 in the illustrated embodiment, etc.), the conversion engine 120 identifies and stores the output coding sequence in memory (e.g., the memory 204, etc.). The conversion engine 120 then returns, as indicated in FIG. 3A, to step 310 and proceeds with the next temperature (e.g., the next N-th temperature, etc.) in the temperature data structure (e.g., 5, 3, 1, 0.1, 0.01, etc.). At the next temperature in the temperature data structure (e.g., per the temperature, annealing, or cooling schedule, etc.), the conversion engine 120 proceeds consistent with the above description of steps 312-322.

When the steps 312-322 are repeated and completed for the final temperature, i.e., the N-th temperature, the conversion engine 120 generates and returns a report, at 324, which includes at least a portion of the coding sequences generated at 314 and corresponding scores and other information about the coding sequences (e.g., the position of inverted repeats, deleterious motifs, miRNA target sites, etc.). The conversion engine 120 may return the report to a user associated with the method 300 and/or store the report in memory (e.g., the memory 204 associated with the conversion engine 120, etc.). Thereafter, the user may implement one or multiple of the coding sequences, generated at 314, and included in the report, in the validation phase 140 of FIG. 1.

FIGS. 4A-F illustrate an example method 400 for use in enhancing translation and/or protein expression for a gene of interest, by way of the parallel tempering process. The method 400 is described in connection with the example system 100 of FIG. 1 and the example computing device 200 of FIG. 2. However, it should be appreciated that the method 400 is not limited to the system 100 or the computing device 200, but may be implemented in a variety of different systems and/or computing devices. Likewise, the systems and computing devices described herein should not be understood to be limited to the example method 400, or other methods described herein.

The method 400 is described herein with reference to an example coding sequence, and where certain example coding sequence parameters are applied (for illustrative purposes). In particular, in the method 400, the following coding sequence parameters are used (e.g., as part of initialization, etc.): GC content above about 56% and below about 60%; data relating to removing inverted repeats; data relating to removing miRNA target sites; data relating to removing deleterious motifs; parameters specifying no sequence redundancy longer than 18nt with previous coding sequence output; parameters specifying twenty-five iterations per swap, four hundred swaps, and eight chains; an annealing or temperature schedule for simulated annealing (or parallel tempering): T=(1, 2, 4, 7, 14, 27, 52, 100); and each temperature cycle being run for about one hundred iterations. It should be appreciated that these parameters are merely illustrative and may be different in other method embodiments.

At the outset in the method 400, the conversion engine 120 accesses one or multiple data structures which include the initialization parameters (as described above) to be used in the process, a listing and/or identification of the deleterious motifs and miRNA target sties, and the input coding sequence 110, in the appropriate form to be provided to the conversion engine 120. Each of the above may be stored in one or more data structures included in memory (e.g., the memory 204, etc.) of the conversion engine 120 or associated with and/or coupled thereto. The deleterious motifs and miRNA target sites will, in general, be the same as those described above in the method 300 (with reference to FIGS. 3A-C), yet may be dependent on the type of plant which is the target of the method 400 and/or the specific expression(s) identified for the coding sequence.

With that said, as shown in FIG. 4A, once initialized (e.g., once the desired initialization parameters are identified, retrieved, etc.), the conversion engine 120 determinates, at 402, whether the input coding sequence 110 is an amino acid (aa), or not. If the input coding sequence 110 is an amino acid, the conversion engine 120 backtranslates the nucleotide sequence, at 404, to thereby provide a coding sequence, i.e., the input coding sequence, for use in the method 400. This may be done by applying a codon equivalence table to identify which amino acids correspond to which codons, and then a codon frequency table to determine which codons are more common at different positions. In doing so, the conversion engine 120 may provide the same initialization for the method 400, at 404, as provided through random codon initialization (e.g., where the input, at 402, is not an amino acid; etc.), for input to step 406 described below.

After the amino acid is backtranslated to a nucleotide sequence, at 404, or when the input coding sequence is not an amino acid, at 402, the conversion engine 120 initializes a number (N) of sequences, at 406, consistent with the chain parameter imposed on the method 400 (in this example embodiment). As such, in this example (as noted by the above-parameters), the conversion engine 120 initializes eight separate sequences (or chains) of the input coding sequence 110, at 406.

FIG. 4B illustrates a sub-process for initializing the coding sequences, at 406. As shown, the initialization process includes accessing the input coding sequence, from which the conversion engine 120 starts, i.e., the input coding sequence 110, and generating, at 406a, a series of coding sequences (or chains), starting with 1. This may be accomplished by merely copying the input coding sequence 110, at 406b, where there is “no change” to the coding sequence (i.e., duplicate the coding sequence). Additionally, or alternatively, the conversion engine 120 may impose random changes, at 406b, to codons (with equivalent codons) within the input coding sequence 110, thereby initializing a coding sequence different than the original coding sequence. Further, the conversion engine 120 may identify, at 406b, most common equivalent codons in the codon frequency table referenced above and include one or more of the codons in the input coding sequence again to provide a coding sequence different than the original/input coding sequence. That said, in connection with imposing random changes (versus simply utilizing a most common codon), weighted distributions may be utilized to provide increased chances for a codon deemed to be more common than others while still providing at least some chances for more uncommon codons (as opposed to just selecting the more common codon outright). It should be appreciated that the conversion engine 120 may employ one or more various combinations of these mechanisms to initialize the coding sequences in other embodiments.

After the coding sequence is initialized for the first chain, at 406b, the conversion engine 120 returns to 406a, whereby the chain is incremented to C=2, and then the conversion engine 120 initializes the coding sequence for the next chain at 406b (i.e., C=2) through one or more of the mechanisms above. It should be appreciated that the same mechanism for initializing the coding sequence for the specific chain is not necessarily the same for each chain. One chain (e.g., C=1) may initialize a coding sequence as a duplicate of the input coding sequence, while another chain (e.g., C=5) may include a random codon swap to the input coding sequence. When the desired number of chains of coding sequences is initialized (e.g., through C=8, or eight coding sequences in this example embodiment; etc.), the conversion engine 120 returns the eight coding sequences to the method 400 of FIG. 4A, to step 408.

In turn, the conversion engine 120 enters a swap loop, at 408, whereby each of the coding sequences (C=1-8) are swapped between the different temperatures (T) for the annealing schedule in the initialization parameters. In particular, each of the coding sequences is initially assigned to a temperature from the annealing schedule (1, 2, 4, 7, 14, 27, 52, 100). Thereafter, the coding sequences proceed into the swap loop, whereupon the conversion engine 120 provides a parallel process for each of the chains (or coding sequences) separately, for the assigned temperature. Specifically, for example, the conversion engine 120 initializes, at 410, each of eight branches of the swap loop, with each of the chains of coding sequences being assigned to one of the branches, along with the corresponding temperature. Thereafter, for each branch, the conversion engine 120 runs an optimization cycle for the coding sequence and corresponding temperature of the branch, at 412.

Notwithstanding the above, it should be appreciated that the annealing schedule may be dynamic in nature (instead of limited to the eight particular temperatures identified above, or particular temperatures in general). In connection therewith, the annealing schedule may be dynamically calculated using a geometric function, for example, such as represented by Equation 3.

$${( a * {b}^{\frac{1}{k - 1}} )}^{n} ∀ n ∈ [ 0 , k - 1 ] \qquad ( 3 )$$

In Equation 3, for example, a may have a value of 1, b may have a value of 100, and k represents a number of chains used by the parallel tempering process of method 400. In this manner, Equation 3 may be used to generate a set of k different numbers between 1 and 100, which will be more clustered together towards one side and more sparse towards the other side. For instance, for a system with eight chains (as in method 400), the values generated would be [1.0, 1.93069772888325, 3.72759372031494, 7.196856730011519, 13.894954943731374, 26.82695795279725, 51.7947467923121, 99.99999999999997](where the values may then be rounded to the nearest integer for simplicity) (which coincidentally result in the temperatures in the annealing schedule parameter above). It should be appreciated that other temperatures may be identified by changes of the variables in Equation 3.

Then in the method 400, within each branch of the swap loop, FIGS. 4C-4E illustrate a sub-process for the optimization cycle (step 412), for the given branch. As shown in FIG. 4C, the conversion engine 120 enters a first iteration (i.e., i=1) for the optimization cycle (for the given branch), at 412a. In this example embodiment, as indicated above, the conversion engine 120 will enter twenty-five iterations for each optimization cycle for each branch (i.e., i=1, i=2, . . . i=25) (i.e., where the number of cycles=25 in FIG. 4C, etc.), but may enter a different number of iterations in other method embodiments (e.g., 15, 50, 100, etc.).

For the first iteration, the conversion engine 120 generates a new coding sequence, at 412b, based on the input coding sequence of the given branch. Specifically, as shown in FIG. 4D, the conversion engine 120 chooses one feature of the coding sequence to modify, based on a probability score for the feature, at 412b-1, and changes one codon that improves that feature of the coding sequence, at 412b-2. In general, the conversion engine 120 determines an impact of multiple of the features or issues with the coding sequence (e.g., GC content, deleterious motifs, inverted repeats, etc.), and then selects one of the features/issues, often the feature/issue with the highest or a relatively high impact on scoring of the coding sequence. As described in more detail below, the scoring of the coding sequence generally includes an issue or feature score f(x) (e.g., as determine based on Equations 1, 6, or 7, etc.), which is weighted, and which is provided in the weighted random probability distribution vector P in Equation 4, below.

$$P = \{ p ( {w}_{1} {f}_{1} ( x ) ) … p ( {w}_{n} {f}_{n} ( x ) ) \} \qquad ( 4 )$$

In connection therewith, the conversion engine 120 identifies each of the specific features or issues of the coding sequence for changing, which are “hits” for the features or issues (e.g., based on the probability distribution vector P, etc.), or identifies each of the features or issues based on the presence of specific substrings in the coding sequence to be removed (e.g., inverted repeats, deleterious motifs, miRNA target sites, etc.) (as identified in the data accessed as the outset of method 400).

For a given coding sequence x and for a given negative feature or issue, the vector H, as defined below, is an expression of the hits (h) of the specific feature or issue in the coding sequence x:

$$H = \{ {h}_{1} , {h}_{2} … {h}_{n} \}$$

What's more, the feature score f(x), as used herein, is then defined as the normalized sum of the length of the hits (h):

$$f ( x ) = {\sum }_{i} \frac{\vert {h}_{i} \vert}{\vert x \vert}$$

In this manner, in accounting for the hits of the issues, for example, f(x) is a function of how far the sequence is from its ideal with respect to the calculated term.

When the above feature or issue expression is combined into Equation 4, a weighted probability distribution is then provided, where the probability of selecting a feature to be improved (at step 412b-1) is directly proportional to its score (S), for example, S(x)=W(x)*f(x) (where f(x), in turn, represents one of the energy scores for a feature or issue, again, from Equations 1, 6, or 7; etc.). As such, the overall score of a given energy term may be calculated as the product of its user defined weight, W(x), multiplied by the energy of the feature or issue, f(x). In general, the change is expected to improve the coding sequence, at least as defined in the scoring below. It should be appreciated that such weights may be selected or omitted based on a feature or issue specific data and/or preferences. It should be further appreciated that other expressions of the coding sequence may be employed to select one or more codons to be changed at steps 412b-1 and 412b-2.

While Equation 4 may be employed alone, in this example embodiment, the conversion engine 120 may further normalize the feature score, f(x). Specifically, the conversion engine 120 may employ a normalization equation, as defined in Equation 5, which brings each score to a number between 0 and 1, and Σᵢp(wᵢfᵢ(x))=1.

$$p ( x ) = \frac{x}{{Σ}_{i} {w}_{i} {f}_{i} ( x )} \qquad ( 5 )$$

Based on the normalized score, the conversion engine 120 then selects a feature or issue (e.g., based on impact to scoring for the coding sequence and/or, potentially, at random; etc.) and changes one codon affecting that selected feature or issue of the coding sequence. As for the particular codon, it may be selected at random, as long as the codon impacts the selected feature or issue, or may be selected based on a frequency or commonality associated with the particular codon.

If, pursuant to the above, for example, the conversion engine 120 selects inverted repeats as the most troublesome (e.g., having a highest feature score, etc.), the conversion engine 120 changes one codon in the coding sequence from the codon frequency table (as defined by the temperature of the branch), to affect and/or eliminate at least one inverted repeat, thereby advancing toward a desired state of the coding sequence. The desired state, then, with regard to inverted repeats and deleterious motifs, is to have zero occurrences of these features or issues. The desired state for GC content is to be within a user defined range, as described above (e.g., between about 56% and about 60%, etc.).

At this point, when the codon is changed, the conversion engine 120 calculates a new sequence score for the coding sequence with the change, at 412c. In so doing, as shown in FIG. 4E, the conversion engine 120 receives the coding sequence (as the input) and then identifies the desired scoring parameters. The conversion engine 120 employs, at 412c-1 and 412c-2, in this example, Equations 6 or 7 below to calculate a score for the coding sequence for the given feature for each of the scoring parameters. In general, as suggested above, the conversion engine 120 may calculate the new sequence score as an energy score consistent with Equation 1 above, or with Equation 6 or 7 below.

$$E ( x ) = max \{ {w}_{1} {f}_{1} ( x ) , {w}_{2} {f}_{2} ( x ) … {w}_{n} {f}_{n} ( x ) \} + ρ {Σ}_{i} {w}_{i} {f}_{i} ( x ) \qquad ( 6 )$$

Consistent with Equation 6, for example, and as discussed above, the conversion engine 120 transforms different features, x (e.g., inverted repeats, miRNA target sites, etc.) (or issues), and weights, w, each into a single numeric value, as part of scalarization. In this example embodiment, while not limiting, the conversion engine 120 employs augmented Chebyshev for scalarization. Further, in this example embodiment, the ρ is a relatively small number, and in Equation 6, in this example embodiment, the first term (i.e., max) prioritizes the feature that has the worst (or in this case, the highest) score at the time the feature (x) is evaluated, while the second term (the sum weighted by ρ) distinguishes between two coding sequences where, even if the highest scoring feature was not improved, another lower scoring feature was. The energy number provided by Equation 6 is then the basis for comparing two different sequences.

While the above illustrates an expression for scoring an identified feature or issue in the coding sequence, f(x), where the scoring accounts for a range, such as, for example, GC content, codon adaptation, secondary structures, etc., the conversion engine 120 may evaluate a particular feature or issue over the entire length of the coding sequence and verify that the feature is within a certain evaluation range. In order to calculate the score of this feature on sequence x, given a range R={rₘᵢₙ, rₘₐₓ}, the conversion engine 120 may employ the following to express the score of a given feature based on the range:

$$if f ( x ) < {r}_{min} , \frac{{r}_{min} - f ( x )}{{r}_{min}}$$

$$if f ( x ) > {r}_{max} , \frac{f ( x ) - {r}_{max}}{1 - {r}_{max}}$$

$$0 otherwise$$

It should be appreciated that, whether as a feature to be removed entirely, or to be included, or to be maintained within a range, the conversion engine 120 may be adjusted, as appropriate, to optimize the specific desired profile of the coding sequence.

That said, more particularly in this example, with reference to FIG. 4E, in calculating the new sequence score (at step 412c-2), the conversion engine 120 accesses the input sequence and user determined scoring parameters, and then employs Equation 7, which is a more specific version of Equation 6 that is specific to the example herein (and to the enzyme Cpf1).

$${E}^{′} = max ( 100 * {f}_{m R N A} , 2.5 * {f}_{IR} , {f}_{M o t i f s} , 100 * {f}_{redundancy} ( C p f {1}_{n e w} , C p f {1}_{Reference} ) ) + 0.001 * ( 100 * {f}_{m R N A} + 2.5 * {f}_{IR} + {f}_{M o t i f s} + 100 * {f}_{redundancy} ( C p f {1}_{n e w} , C p f {1}_{Reference} ) ) \qquad ( 7 )$$

When the score is determined for the coding sequence, the conversion engine 120 determines, at 412d, in FIG. 4C, whether the score for the coding sequence is higher than the score for the prior coding sequence (i.e., the input at 412a). When the score indicates an improvement, the changed coding sequence is accepted and saved, at 412e, along with the score in memory (e.g., the memory 204 associated with the conversion engine 120, etc.) as the current coding sequence for the branch. The conversion engine 120 returns to 412a, where the conversion engine 120 advances to the next iteration (e.g., i=2, etc.), and in this next iteration, the current coding sequence (i.e., the new coding sequence accepted at 412e) is used as the starting coding sequence point for generating the new sequence, at 412a.

Conversely, if the score does not reflect enhancement, the conversion engine 120 applies Equation 2 above to determine a probability between the scores, based on the temperature, and then compares the probability to a randomly generated threshold (e.g., between 0 and 1, etc.). That said, it should be appreciated that other manners of expressing the performance of the coding sequence (whether based on temperature or not) may be employed relative to one or more thresholds.

When the probability satisfies the random threshold, the new coding sequence is accepted, at 412f, by the conversion engine 120, and saved in memory (e.g., the memory 204, etc.), and returned as the input coding sequence at 412a for the next iteration (e.g., i=2, etc.). Conversely, when the calculated probability does not satisfy the random threshold, the new coding sequence is rejected, at 412f, and the prior input coding sequence is reused as the input coding sequence at 412a for the next iteration (e.g., i=2, etc.). In this manner, it is permitted, based on the randomly selected threshold, for a lower score coding sequence to be advanced in the branch as a starting coding sequence for the next iteration, thereby providing randomness and flexibility into the method 400.

Additionally, or alternatively, the method 400 may further be employed to add features that are desired to be included in the coding sequence. Examples of such features may include, for example, motifs associated with increased expression or stability. In order to calculate the score of a feature that intends to increase or even to maximize the number of times a certain motif M appears in the coding sequence x, Equation 8 may be used.

$$1 - \frac{f ( x , M )}{f ( {x}_{U} , M )} \qquad ( 8 )$$

In Equation 8, f(x, M) is the number of times that desired motif M appears in coding sequence x, and x_{U} indicates a codon optimized version of the coding sequence S designed in such a way that it contains a maximum or near maximum number of the desired motifs M encoded therein. As such the score will be a minimum of zero when S=S_{U} and a maximum of one when the coding sequence contains no occurrences of motif M.

In order to determine f(x_{U}, M), the example of four-nucleotide desired motifs, M=ACGT, is provided. When included into a coding sequence, the desired motif M may be implemented in three possible reading frames depending on the relative position of the motif to the start codon.

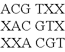

Thereafter, the conversion engine 120 identifies possible amino acids that encode for the above combinations. For example, for the first example ACG TXX, the conversion engine 120 determines that the combination may be encoded as amino acids TC, TS, TY, TL, TF, and TW. The conversion engine 120 repeats the same determination for the second and third reading frames. The conversion engine 120 then determines that motif ACGT can be potentially encoded by a set of amino acids A={TC, TS, TY, TL . . . }. Consequently, the coding sequence x may be translated into amino acid sequence x_{A}. The conversion engine 120 is then able to process a number of times the amino acids in set A (which again, are the amino acid forms of motif M) are contained in coding sequence x_{A}. This number will be equal to the maximum number of times motif M can be contained in coding sequence x, and thus equal to f(x_{U}, M). With this term, the conversion engine 120 is permitted to evaluate, in various example embodiments, the presence of the desired feature in scoring the input and output coding sequences of the different iterations in FIGS. 4C and 4E.

It should be appreciated that, based on the above, apart from removing specific features or issues, certain features may be added to the coding sequence, through method 400, with the effect of the addition and/or the prevalence of the desired feature indicated as expressed above in Equation 8 (which may be included in Equation 6 and/or Equation 7). That said, it should be appreciated that in various embodiments, the desired feature(s) scoring and/or evaluation may be omitted, as above, where only the removal of the features and/or issues is the focus of the method 400.

When all of the iterations (e.g., i=1 through i=25 in this example embodiment, etc.) are complete (i.e., when the optimization cycle is over), the conversion engine 120 returns the one coding sequence for each of the branches into FIG. 4A, along with metrics, scores, etc. related to the coding sequences. In connection therewith, the conversion engine 120 then joins, at 414, the eight different branches together, thereby considering the candidate coding sequences, from each of the branches, together, yet each still associated with one of the temperatures in the annealing schedule (as assigned to the branch which provided the coding sequence).

In connection therewith, the conversion engine 120 enters a chain swap, at 416. As part of the chain swap, the conversion engine 120 exchanges the candidate coding sequences among the different branches, whereby each output coding sequence from one temperature is exposed to an optimization cycle at a different temperature. In particular, the conversion engine 120 attempts, for example, to assign the lower scoring coding sequences (i.e., better) to lower temperatures, while assigning the higher scoring coding sequences (i.e., worse) to higher temperatures. The higher temperatures, accordingly, permit more variability and flexibility whereby the worse scoring coding sequences are eligible for greater change (e.g., to search more sequences, etc.), while the better scoring coding sequences are exposed to lower temperatures which permit less variability (e.g., to provide refinement, etc.). The detail of the chain swap is shown in FIG. 4F. The conversion engine 120 employs Equation 9 for one of the chains (e.g., C1, C2, . . . Cn, etc.) (e.g., the highest temperature chain, etc.), where ΔE is difference in energies between the two chains and Δβ=1/(T₁−T₂) is the difference in the reciprocal of the temperatures between the two chains.

$$p = ( 1 , {e}^{Δ E Δ β} ) \qquad ( 9 )$$

In addition to the probability (p) determined in Equation 9, the conversion engine 120 determines a random number (e.g., between 0 and 1, etc.) and compares the probability to the random number. When the probability exceeds the random number, the conversion engine 120 then swaps the coding sequence from the chain, at 416b, with a coding sequence from another chain. The number of swaps performed may be a user provided (or user defined) parameter (e.g., with a default of 100, etc.). The starting chain, at iteration zero, may depend on the initialization mode selected by the user (see, FIG. 4B). At any swap point different from zero, the sequence a chain has is equal to the sequence that was obtained during the last iteration of the optimization algorithm prior to the swap. In connection therewith, example embodiments of the present disclosure may favor swaps that send worse sequences to higher temperatures through Equation 9. If a higher temperature chain 1 (A/3=1/(higher_temperature)−1/(lower temperature) will be negative) has a higher performing sequence (ΔE=lower−higher score=negative) then p=(1,e\hat{ }Positive)=(1, big_number)=1, the swap will always happen. On the other hand if the higher temperature chain has a worse scoring sequence (Δβ=1/higher−1/lower will be negative) (ΔE higher−lower score=positive) then p=(1, e\hat{ }negative)=(1, smaller than_1), the swap may be rejected.

Once the swap of the candidate coding sequences is complete, as shown in FIG. 4A, the conversion engine 120 returns to 408, whereby the conversion engine 120 increments the swap counter (j) from 1 to 2, for example, and proceeds as described above. The conversion engine 120 will continue in method 400 until a number of swaps reaches a predefined maximum (e.g., 100, 400, etc.) or the algorithm reaches an early stopping condition. An example stop condition may include where all the penalties for issues of features in Equation 7 are reduced to zero by the changing of codons within the coding sequence, etc. In this example, there is no longer a basis to select a specific feature and/or change a codon in the content of the method 400. It should be appreciated that other stop condition may exist, as defined, for example, by a user associated with the method 400 or the specific desires associated with the expression to be enhanced through method 400.

Finally, in the method 400, the conversion engine 120 generates and returns a report, which includes one or more of the candidate coding sequences generated as part of the optimization cycle, at 412, and corresponding scores and other information about the coding sequences (e.g., the position of inverted repeats, deleterious motifs, miRNA target sites, etc.). The conversion engine 120 may return the report to a user associated with the method 400 and/or store the report in memory (e.g., the memory 204 associated with the conversion engine 120, etc.). Thereafter, the user may implement one or multiple of the coding sequences, generated in the method 400, and included in the report, in the validation phase 140 of FIG. 1. Thereafter, the user may implement one or multiple of the coding sequences included in the report, in the validation phase 140 of FIG. 1.

In this manner, the systems and methods herein provide a multiple chain Monte Carlo (MCMC) approach to sequence codon optimization, while providing confidence in the process and permitting the easy implementation of additional optimization factors. What's more, the systems and methods herein, especially as to FIG. 4, may be employed for other applications other than expression enhancement, like tissue specificity or transcript stability, etc.

In view of the above, the systems and methods herein provide for transformation of a given sequence, where the amino acids are not changed, while the DNA sequence is. Specifically, the systems and methods herein transform gene coding sequences to improve or change the RNA or protein expression level of the gene. The process includes, in summary, and as explained above, altering codon usage (e.g., in a position-specific fashion, etc.) to resemble the target species' coding sequences, altering the AT:GC ratio, removing or adding RNA structures, removing or adding regulatory sequence motifs, removing or adding runs of common or rare codons, adding Kozak sequences, and adding or removing inverted repeats. As described, the systems and method herein provide for a very large number of possible solutions to be generated from the same input sequence, thus creating sequence diversity.

Again and as previously described, it should be appreciated that the functions described herein, in some embodiments, may be described in computer executable instructions stored on a computer readable media, and executable by one or more processors. The computer readable media is a non-transitory computer readable storage medium. By way of example, and not limitation, such computer-readable media can include RAM, ROM, EEPROM, CD-ROM or other optical disk storage, magnetic disk storage or other magnetic storage devices, or any other medium that can be used to carry or store desired program code in the form of instructions or data structures and that can be accessed by a computer. Combinations of the above should also be included within the scope of computer-readable media.

It should also be appreciated that one or more aspects of the present disclosure transform a general-purpose computing device into a special-purpose computing device when configured to perform the functions, methods, and/or processes described herein.

As will be appreciated based on the foregoing specification, the above-described embodiments of the disclosure may be implemented using computer programming or engineering techniques including computer software, firmware, hardware or any combination or subset thereof, wherein the technical effect may be achieved by performing at least one of the operations described herein and/or recited in the corresponding claims hereinafter. For example, the technical effect may be achieved by a) for an input coding sequence, identifying a feature of the coding sequence as a candidate for change, based on an effect of the feature on a score for the coding sequence; b) selecting, by a conversion engine computing device, a codon of the coding sequence associated with the identified feature; c) inserting a codon equivalent for the selected codon, wherein the codon equivalent is based on a temperature associated with the coding sequence; d) calculating a score for the coding sequence with the codon equivalent and a score for the input coding sequence; and e) advancing the coding sequence with the codon equivalent to a next iteration when the score for the coding sequence with the codon equivalent indicates an enhancement relative to the score of the input coding sequence.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Specific values disclosed herein are example in nature and do not limit the scope of the present disclosure. The disclosure herein of particular values and particular ranges of values for given parameters are not exclusive of other values and ranges of values that may be useful in one or more of the examples disclosed herein. Moreover, it is envisioned that any two particular values for a specific parameter stated herein may define the endpoints of a range of values that may be suitable for the given parameter (i.e., the disclosure of a first value and a second value for a given parameter can be interpreted as disclosing that any value between the first and second values could also be employed for the given parameter). For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, and 3-9.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When a feature is referred to as being “on,” “engaged to,” “connected to,” “coupled to,” “associated with,” “in communication with,” or “included with” another element or layer, it may be directly on, engaged, connected or coupled to, or associated or in communication or included with the other feature, or intervening features may be present. As used herein, the term “and/or” and the phrase “at least one of” include any and all combinations of one or more of the associated listed items.

Although the terms first, second, third, etc. may be used herein to describe various features, these features should not be limited by these terms. These terms may be only used to distinguish one feature from another. Terms such as “first,” “second,” and other numerical terms when used herein do not imply a sequence or order unless clearly indicated by the context. Thus, a first feature discussed herein could be termed a second feature without departing from the teachings of the example embodiments.

None of the elements recited in the claims are intended to be a means-plus-function element within the meaning of 35 U.S.C. § 112(f) unless an element is expressly recited using the phrase “means for,” or in the case of a method claim using the phrases “operation for” or “step for.”

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A computer-implemented method for use in providing gene translation and protein expression, the computer-implemented method comprising:
identifying, by a conversion engine computing device, a distinct one of a plurality of coding sequences as an input sequence for each of M chains;
for each of the M chains, where M is an integer greater than 1, initializing a defined N iteration(s) in parallel for the input coding sequence for the chain, where each chain is assigned a different one of multiple defined temperatures in a temperature schedule, and where N is an integer greater than 1; and
for each of the N iterations:
(a) modifying the input coding sequence by changing a first codon of the input coding sequence to be an alternate, but equivalent, codon as defined by the assigned one of the defined temperatures;
(b) calculating a score for the modified coding sequence based on inclusion of features of the modified coding sequence, the features including GC content and multiple undesirable features, which include deleterious motifs, miRNA target sites, and/or inverted repeats;
(c) advancing the modified coding sequence to a next iteration based on the calculated score being better than a score of the input coding sequence and the iteration being less than N; and
(d) identifying the modified coding sequence as an output coding sequence for the N iterations based on the calculated score being better than the score of the input coding sequence and the iteration being equal to N;
after the N iterations, for each of the M chains, swapping, by the conversion engine computing device, the output coding sequence of said chain with the output coding sequence of a different one of the M chains;
initializing another N iteration(s) for the output coding sequence as the input coding sequence at one of the multiple defined temperatures in the temperature schedule and, for each of the another N iteration(s), repeating operations (a) through (d);
after swapping the output coding sequences among the M chains X times, identifying, by the conversion engine computing device, at least one of the output coding sequences as a final coding sequence; and
genetically modifying a plant, via an expression cassette including the final coding sequence, by introducing the expression cassette including the final coding sequence into the genome of the plant.

2. The computer-implemented method of claim 1, further comprising, for each of the N iterations, identifying one of the features of the input coding sequence and then identifying said codon as associated with the identified one of the features, prior to modifying the input coding sequence to include the alternate, but equivalent, codon.

3. The computer-implemented method of claim 2, wherein identifying the one of the feature includes identifying the one of the features based on an impact of the one of the features on the score for the input coding sequence.

4. The computer-implemented method of claim 1, wherein calculating the score for the modified coding sequence includes calculating the score based on:
$$p = \{ p ( {w}_{1} {f}_{1} ( x ) ) … p ( {w}_{n} {f}_{n} ( x ) ) \} ;$$
where each of w₁ through wₙ is a weighting factor; and
wherein each of f₁(x) through fₙ(x) is representative of a different one of said features of the coding sequence.

5. (canceled)

6. The computer-implemented method of claim 1, wherein advancing the modified coding sequence to a next iteration includes advancing the modified coding sequence to the next iteration based on the calculated score satisfying a threshold, and based on the iteration being less than N; and
wherein the threshold includes one of a static threshold and a randomly generated threshold per iteration.

7. The computer-implemented method of claim 6, wherein advancing the modified coding sequence is based on a probability defined by a probability algorithm satisfying the randomly generated threshold;
wherein the probability algorithm includes:
$$p = exp ( - \frac{{E}^{′} - E}{τ} ) ;$$
wherein E is the calculated score of the input coding sequence, E′ is the calculated score of the modified coding sequence, and T is the one of the defined temperatures.

8. The computer-implemented method of claim 1, wherein M is less than ten and wherein N is less than 100.

9.-18. (canceled)

19. A computer-implemented method for use in providing gene translation and protein expression, the computer-implemented method comprising:
identifying, by a conversion engine computing device, a distinct one of a plurality of coding sequences as an input sequence for each of M chains;
for each of the M chains, where M is an integer greater than 1, initializing a defined N iteration(s) in parallel for the input coding sequence for the chain, where each chain is assigned a different one of multiple defined temperatures, and where N is an integer greater than 1; and
for each of the N iterations:
(a) modifying the input coding sequence by changing a first codon of the input coding sequence, to be an alternate, but equivalently-coding, codon as defined by the assigned one of the defined temperatures;
(b) calculating a score for the modified coding sequence based on inclusion of features of the modified coding sequence, the features including GC content and multiple undesirable features, which include deleterious motifs, miRNA target sites, and/or inverted repeats;
(c) advancing the modified coding sequence to a next iteration based on the calculated score being better than a score of the input coding sequence and the iteration being less than N; and
(d) identifying the modified coding sequence as an output coding sequence for the N iterations based on the calculated score being better than the score of the input coding sequence and the iteration being equal to N;
after the N iterations, for each of the M chains, swapping, by the conversion engine computing device, the output coding sequence of said M chain with the output coding sequence of a different one of the M chains;
initializing another N iteration(s) for the output coding sequence as the input coding sequence at one of the multiple defined temperatures in the temperature schedule and, for each of the another N iteration(s), repeating operations (a) through (d);
after swapping the output coding sequences among the M chains X times, identifying, by the conversion engine computing device, at least one of the output coding sequences as a final coding sequence; and
genetically modifying a plant, via one or more of a gene gun, electroporation, zinc finger nucleases (ZFNs), transcription activator-like effector nucleases (TALENs), a CRISPR/Cas9 system, and/or a CRISPR/Cpf1 system, to include the final coding sequence.
